(19) United States
(12) Patent Application Publication
(10) Pub. No.: US 2022/0002700 A1
(43) Pub. Date: Jan. 6, 2022

(54) COMPUTATIONALLY-OPTIMIZED ACE2 PEPTIDES FOR COMPETITIVE INTERCEPTION OF SARS-CoV-2

(71) Applicant: Massachusetts Institute of Technology, Cambridge, MA (US)

(72) Inventors: Pranam CHATTERJEE, Cambridge, MA (US); Raghava Manvitha PONNAPATI, Cambridge, MA (US); Eyal PERRY, Cambridge, MA (US); Joseph M. JACOBSON, Newton, MA (US)

(21) Appl. No.: 17/222,676

(22) Filed: Apr. 5, 2021

Related U.S. Application Data
(60) Provisional application No. 63/005,006, filed on Apr. 3, 2020.

(51) Int. Cl. C12N 9/48; A61K 38/48

(52) U.S. Cl. CPC C12N 9/485; C12Y 304/17023; A61K 38/4813

(57) ABSTRACT

Methods and compositions relating to an engineered peptide capable of binding to a biological molecule for viral inhibition. The engineered peptide is computationally-derived from soluble angiotensin-converting enzyme 2 (sACE2), a known receptor for viral spike proteins. The engineered peptide is optimized for minimal size and off-target effects. The engineered sACE2 peptide variants are a suitable targeting domain for fusion proteins of various effects.

### CROSS-REFERENCE TO RELATED APPLICATION

The present application claims the benefit of U.S. Provisional Application No. 63/005,006, filed Apr. 3, 2020, which is hereby incorporated herein in its entirety by reference.

### FIELD OF INVENTION

The present disclosure relates to computational design of viral-competitive peptides, particularly sACE2-derived peptides for targeting SARS-CoV-2, more particularly sACE2-derived peptides for binding to a SARS-CoV-2 spike protein receptor binding domain.

### BACKGROUND

SARS-CoV-2 has emerged as a highly pathogenic coronavirus and has nowspread to over 200 countries, infecting over 50 million people worldwide and killing over 1 million people as of October 2020. Economies have crashed, travel restrictions have been imposed, and public gatherings have been canceled, all while a sizeable portion of the human population remains quarantined. Rapid transmission dynamics as well as a wide range of symptoms, from a simple dry cough to pneumonia and death, are common characteristics of coronavirus disease 2019 (COVID-19) [Wu, J. T. et al. Estimating clinical severity of COVID-19 from the transmission dynamics in Wuhan, China. Nat. Med. 26, 506-510 (2020).]. With no cures readily available [Lurie, N., Saville, M., Hatchett, R. & Halton, J. Developing covid-19 vaccines at pandemic speed. N. Engl. J. Med. 382, 1969-1973 (2020)], and only limited vaccine availability, there is a pressing need for robust and effective therapeutics targeting the virus.

Numerous antiviral strategies have been proposed to limit SARS-CoV-2 replication by preventing viral infection and synthesis [Senanayake, S. L. Drug repurposing strategies for COVID-19. Future Drug Discov. 2 (2020).]. As SARS-CoV-2 is a positive-sense RNA virus, Abbott, et al. recently devised a CRISPR-Cas13d based strategy, termed PAC-MAN, to simultaneously degrade the positive-sense genome and viral mRNAs [Abbott, T. R. et al. Development of CRISPR as an antiviral strategy to combat SARS-CoV-2 and influenza. Cell 181, 865-876.e12 (2020)]. While this method may serve as a potential prophylactic treatment, introducing foreign and relatively large components such as Cas13 enzymes into human cells in vivo presents various delivery and safety challenges [Lino, C. A., Harper, J. C., Carney, J. P. & Timlin, J. A. Delivering CRISPR: a review of the challenges and approaches. Drug Deliv. 25, 1234-1257 (2018).].

Since the 2003 SARS epidemic, it has been widely known that the angiotensin-converting enzyme 2 (ACE2) receptor is critical for SARS-CoV entry into host cells [Du, et al., “The spike protein of sars-cov a target for vaccine and therapeutic development”, Nat. Rev.Microbiol. (2009)].

ACE2 is a monocarboxypeptidase, widely known for cleaving various peptides within the renin-angiotensin system [Tipnes, et al., “A human homolog of angiotensin-converting enzyme: cloning and functional expression as a captopril-insensitive carboxypeptidase”, Journal of Biological Chemistry (2000)]. Functionally, there are two forms of ACE2. The full-length ACE2 contains a structural transmembranedomain, which anchors its extracellular domain to the plasma membrane [Du, et al., “Thespike protein of cars-cov—a target for vaccine and therapeutic development”, Nat. Rev. Alicrobiol. (2009)]. The extracellular domain has been demonstrated as a receptor for the spike (S) protein of SARS-CoV, and recently, for the SARS-CoV-2.

The COVID-19 pandemic, caused by the novel coronavirus SARS-CoV-2, has elicited a global health crisis of catastrophic proportions. With only a few vaccines approved for early or limited use, there is a critical need for effective antiviral strategies.

### SUMMARY

Disclosed herein is a unique antiviral platform, through computational design of ACE2-derived peptides which may target both a viral spike protein receptor binding domain (RBD) and recruit E3 ubiquitin ligases for subsequent intracellular degradation of SARS-CoV-2, or other viruses, in the proteasome. The engineered peptide fusions of the present disclosure demonstrate robust RBD degradation capabilities in human cells and are further capable of inhibiting infection-competent viral production.

In some aspects, the engineered peptide capable of binding to a biological molecule for viral inhibition comprises an sACE2 peptide variant having an amino acid sequence comprising QAKTFLDKFNHEAEDLFY. In some aspects, the engineered peptide capable of binding to a biological molecule for viral inhibition comprises an sACE2 peptide variant having an amino acid sequence comprising AMVDQAWDAQRIFKEAEKFFVSVGLPNMTQGFWENSMLTDPGNVQKAVCHPT AWDLGKGDFRILMCTKVT

In some aspects, the sACE2 peptide variant comprises an amino acid sequence selected from the group consisting of sACE2 peptide variant 1 (SEQ ID NO:1), sACE2 peptide variant 2 (SEQ ID NO:2), sACE2 peptide variant 3 (SEQ ID NO:3), sACE2 peptide variant 4 (SEQ ID NO:4), sACE2 peptide variant 5 (SEQ ID NO:5), sACE2 peptide variant 6 (SEQ ID NO:6), sACE2 peptide variant 7 (SEQ ID NO:7), sACE2 peptide variant 8 (SEQ ID NO:8), sACE2 peptide variant 9 (SEQ ID NO:9), sACE2 peptide variant 10 (SEQ ID NO:10), sACE2 peptide variant 11 (SEQ ID NO:11), sACE2 peptide variant 12 (SEQ ID NO:12), sACE2 peptide variant 13 (SEQ ID NO:13), sACE2 peptide variant 14 (SEQ ID NO:14), sACE2 peptide variant 16 (SEQ ID NO:16), sACE2 peptide variant 17 (SEQ ID NO:17), sACE2 peptide variant 18 (SEQ ID NO:18), sACE2 peptide variant 19 (SEQ ID NO:19), sACE2 peptide variant 20 (SEQ ID NO:20), sACE2 peptide variant 21 (SEQ ID NO:21), sACE2 peptide variant 22 (SEQ ID NO:22), sACE2 peptide variant 24 (SEQ ID NO:24), sACE2 peptide variant 26 (SEQ ID NO:26), sACE2 peptide variant 27 (SEQ ID NO:27), sACE2 peptide variant 28 (SEQ ID NO:28), sACE2 peptide variant 29 (SEQ ID NO:29), sACE2 peptide variant 30 (SEQ ID NO:30), and sACE2 peptide variant 31 (SEQ ID NO:31).

In some aspects, the sACE2 peptide variant comprises an amino acid sequence selected from the group consisting of sACE2 peptide variant 15 (SEQ ID NO:15), sACE2 peptide variant 23 (SEQ ID NO:23) and sACE2 peptide variant 25 (SEQ ID NO:25).

In some aspects, the sACE2 peptide variant comprises an amino acid between 18 and 30 amino acids in length.

In some aspects, the sACE2 peptide variant is capable of binding with a spike protein receptor. In some aspects, the sACE2 peptide variant is capable of binding with a spike protein receptor that is part of a viral envelope. In some aspects, the spike protein receptor is part of at least one coronavirus. In some aspects, the at least one coronavirus is SARS-CoV-2.

In some aspects, the sACE2 peptide variant is capable of fusing to an ubiquitin ligase recruiting domain. In some aspects, the sACE2 peptide variant has a C-terminus, and the ubiquitin ligase recruiting domain is fused to the C-terminus.

In some aspects, a pharmaceutical composition comprises at least one of the sACE2 peptide variants. In some other aspects, the pharmaceutical composition comprises two or more of the sACE2 peptide variants, for simultaneous, sequential or separate administration.

The present disclosure also relates to a method of treating a viral infection in a subject using one or more of the sACE2 peptide variants. In some aspects, the one or more sACE2 peptide variants is provided as a pharmaceutical composition that can be provided to an animal subject, preferably a human subject, wherein the sACE2 peptide variant has an amino acid sequence comprising QAKTFLDKFNHEAEDLFY or AMVDQAWDAQRIFKEAEKFFVSVGLPNMTQGFWENSMLTDPGNVQKA VCHPTAWDLGKGDFRILMCTKVT.

In some preferred aspects, the sACE2 peptide variant comprises sACE2 peptide variant 2 (SEQ ID NO:2).

### BRIEF DESCRIPTION OF THE DRAWINGS

Other aspects, advantages and novel features of the invention will becomemore apparent from the following detailed description of the invention when considered in conjunction with the accompanying drawing, wherein:

FIG. 1 is a rendering of a computationally-engineered sACE2 variant stablybound to the SARS-CoV-2 S protein RBD, according to embodiments of the present disclosure. The mutant was derived from PDB 6MOJ in Rosetta, and was visualized using PyMol.

FIG. 2 is a flowchart of an example method generating a computationally-engineered sACE2 variant, according to embodiments of the present disclosure.

### DETAILED DESCRIPTION

The object of the present disclosure relates to antiviral molecules, and more particularly relates to computational design of viral-competitive peptides. The present disclosure parituclalry relates to engineered sACE2-derived peptides for targeting SARS-CoV-2, more particularly sACE2-derived peptides for binding to a SARS-CoV-2 spike protein receptor binding domain.

Disclosed herein is a computationally truncated and engineered human ACE2 receptor sequence to potently bind to the SARS-CoV-2 RBD. Further disclosed is an optimized peptide variant that enables robust degradation of RBD-sfGFP complexes in human cells, both in trans and in cis with human E3 ubiquitin ligases. The fusion constructs disclosed herein inhibit the production of infection-competent viruses pseudotyped with the full-length S protein of SARS-CoV-2.

The most rapid and acute method of protein degradation intracellularly is at the post-translational level. Specifically, E3 ubiquitin ligases can tag endogenous proteins for subsequent degradation in the proteasome [Ardley, H. C. & Robinson, P. A. E3 ubiquitin ligases. Essays Biochem. 41, 15-30 (2005).]. Thus, by employing guiding E3 ubiquitin ligases with viral-targeting peptides, the objects and methods of the present disclosure can mediate depletion of SARS-CoV-2 viral components in vitro.

Herein, a targeted intracellular degradation strategy for SARS-CoV-2 is disclosed by computationally designing peptides that bind to its spike (S) protein receptor binding domain (RBD) and recruit a E3 ubiquitin ligase for subsequent proteasomal degradation. The disclosed results of example experiments identify an optimal peptide variant that mediates robust degradation of the RBD fused to a stable superfolder-green fluorescent protein (sfGFP) in human cells and inhibits infection-competent viral production [Pédelacq, J.-D., Cabantous, S., Tran, T., Terwilliger, T. C. & Waldo, G. S. Engineering and characterization of a superfolder green fluorescent protein. Nat. Biotechnol. 24, 79-88 (2005).].

The soluble form of ACE2 lacks the membrane anchor, thus preserving binding capacity, and circulates in small amounts in the blood [Wysocki, et al. “Targeting the degradation of angiotensin ii with recombinant angiotensin-converting enzyme 2. Prevention of angiotensin ii-dependent hypertension”, Hypertension (2010)]. Overexpression of soluble ACE2 (sACE2) may act as a competitive interceptor of SARS-CoV-2 and other coronavinises by preventing binding of the viral particle to the endogenous ACE2 transmembrane protein.

sACE2, however, is capable of binding other biological molecules in vivo,such as integrins [Clarke, et al., “Angiotensin Converting Enzyme (ACE) and ACE2 Bind Integrins and ACE2 Regulates Integrin Signaling”, PLOS ONE (2012)]. It is therefore an object of the present disclosure to ensure therapeutics targeting SARS-CoV-2 epitopes withstand the possibility of viral mutation, which may allow the virus to overcome the host adaptive immune response [Andersen, K. G., Rambaut, A., Lipkin, W. I., Holmes, E. C. & Garry, R. F. The proximal origin of SARS-CoV-2. Nat. Med. 26, 450-452 (2020)]. Embodiments of the present disclosure relate to engineering minimal sACE2 peptides, such as by in silico protein modeling, that not only maintain potent RBD binding, but also possess reduced off-target interaction with the integrin α5β1 receptor.

Furthermore, sACE2 preserves the peptidase activity of its transmembrane counterpart, and its overexpression can thus interfere with pathways dependent on this function, such as insulin/Akt signaling [Zhong, et al., “Enhanced angiotensin converting enzyme 2 regulates the insulin/Akt signalling pathway by blockade of macrophage migration inhibitory factor expression”, Br. J. Pharmacol. (2008)].

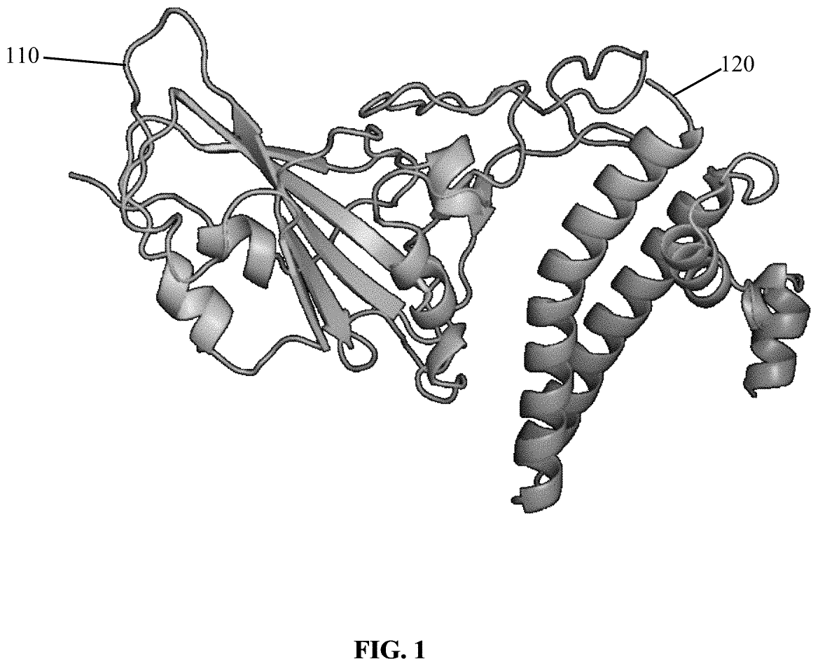

In certain embodiments, the present disclosure is directed to engineered sACE2 peptide variants that lack catalytic activity and differentially bind to the SARS-CoV-associated spike (S) protein,for reduced off-target effects during competitive interception of SARS-CoV-2. Recently, the co-crystal structure of the SARS-CoV-2 spike protein RBD bound to the sACE2 receptor was published [Lan, et al., “Crystal structure of the 2019-nCoV spike receptor-binding 2 domain bound with the ACE2 receptor”, bioRxiv (2020)]. For the present purpose, the associated Protein Data Bank (PDB) entry, 6M0J, can be utilized to model the interface energy between the RBD and sACE2. Specifically, high-throughput in silico truncations to the N-terminal peptidase domain of ACE2 can be conducted, as well as associated structural motifs, using the Rosetta protein modeling software [Rohl, et al., “Protein structure prediction using Rosetta”, Methods Enzymol. (2004)]. This can minimize the ACE2 protein structure to the sufficient components needed for binding to the RBD (FIG. 1).

FIG. 1 is a rendering of an exemplary computationally-engineered sACE2 variant 110 stably bound to the SARS-CoV-2 S protein RBD 120. The mutant was derived from PDB 6MOJ in Rosetta, and was visualized using PyMol.

Methods and products according to the present disclosure have numerous advantages, including but not limited to, both of the peptide and E3 ubiquitin ligase components can be engineered from endogenous human proteins, which may reduce the risk of immunogenicity.

Also, the peptide fusion platform as a prophylactic provides a viable alternative to current antiviral strategies being explored for COVID-19 and other viruses. Antiretroviral protease inhibitors for HIV, such as lopinavir and ritonavir, have shown minimal efficacy in clinical trials of COVID-19, and generated adverse effects in a subsection of patients [Cao, B. et al. A trial of lopinavir-ritonavir in adults hospitalized with severe covid-19. N. Engl. J. Med. 382, 1787-1799 (2020).]. Similarly, antimalarials, such as hydroxychloroquine and chloroquine, which may glycosylate ACE2, have demonstrated no benefit in patients infected with SARS-CoV-2 in randomized, controlled studies [Boulware, D. R. et al. A randomized trial of hydroxychloroquine as postexposure prophylaxis for covid-19. N. Engl. J. Med. 383, 517-525 (2020).]. A standard timeframe to fully assess safety and efficacy of a vaccine takes well over one year [Callaway, E. The race for coronavirus vaccines: a graphical guide. Nature 580, 576-577 (2020).].

The platform of the present disclosure provides a rapid and direct targeting mechanism, which coupled with its size and human-protein derivation, presents numerous advantages as compared with existing strategies. The strategy of utilizing a computationally designed peptide binder linked to an E3 ubiquitin ligase can be effective not only for SARS-CoV-2, but also for other viruses and drug targets that have known binding partners. With already over 30,000 co-crystal structures currently in the PDB, and structure determination becoming more routine with advances in cryogenic electron microscopy, the computational peptide engineering pipeline presented here provides a versatile new therapeutic platform in the fight against COVID-19, future emergent viral threats, and numerous diseases.

The Peptidrive algorithm [Sedan, et al., “Peptiderive server: derive peptide inhibitors from protein-protein interactions”, Nucleic Acids Research (2016)] can be applied multiple times for each peptide length between 30 and 100 amino acids to find candidates derived from ACE2 which bind to the spike protein with high affinity. Each candidate protein can be computationally relaxed. In embodiments, those with the lowest total energy score, and thus highest binding affinity, can be selected.

In certain embodiments, a Monte Carlo-based multi-scale algorithm called FlexPepDock [Lyskov, et al., “Rosetta FlexPepDock web server—high resolution modeling of peptide-protein interactions”, Nucleic Acids Research (2011)] can be used, in silico docking protocols can be conducted of candidate sACE2 peptides with structure of the α5β1-integrin headpiece (PDB 3V14) [Nagae, et al., “Crystal structure of alpha5beta 1 integrin ectodomain: Atomic details of the fibronectin receptor”, J. Cell. Biol. (2012)], which has demonstrated binding to ACE2 in previous studies [Clarke, et al., “Angiotensin Converting Enzyme (ACE) and ACE2 Bind Integrins and ACE2 Regulates Integrin Signaling”, PLOS ONE (2012)]. By normalizing interface energies, variants with low affinity for the α5β1- integrin can be selected, while binding to the S protein RBD is retained. Additionally, sACE2 variants can be docked to other S proteins from the Coronavirus family, by employing PDB entries of other ACE2-S protein co-structures [Song, et al., “Cryo-EM structure of the SARS coronavirus spike glycoprotein in complex with its host cell receptor ACE2”, PLOS Pathog (2018); Li, et al., “Structure of SARS coronavirus spike receptor-binding domain complexed with receptor”, Science (2005); Wrapp, et al., “Cryo-EM structure of the 2019-nCoV spike in the prefusion conformation”, Science (2020)].

Successful docking to other S proteins can provide additional confidence that selected variants will withstand viral mutations during the course of the outbreak.

The present disclosure includes sACE2-derived protein sequences according to certain embodiments of the present invention, as provided in Table 1.

TABLE 1
sACE2-derived Protein Sequences.

| sACE2 | peptide | |
|---|---|---|
| variant # | length | Sequence |
| 1 | 18 | QAKTFLDKFNHEAEDLFY |
| 2 | 23 | QAKTFLDKFNHEAEDLFYQSSLA |
| 3 | 23 | IEEQAKTFLDKFNHEAEDLFYQS |
| 4 | 25 | STIEEQAKTFLDKFNHEAEDLFYQS |
| 5 | 28 | STIEEQAKTFLDKFNHEAEDLFYQSSLA |
| 6 | 29 | STIEEQAKTFLDKFNHEAEDLFYQSSLAS |
| 7 | 30 | STIEEQAKTFLDKENHEAEDLEYQSSLASW |
| 8 | 44 | STIEEQAKTFLDKENHEAEDLFYQSSLASWNYNTNITEENVQNM |
| 9 | 56 | QAKTFLDKFNHEAEDLFYQSSLASWNYNTNITEENVQNMN |
| | | NAGDKWSAFLKEQSTL |
| 10 | 57 | TFLDKFNHEAEDLFYQSSLASWNYNTNITEENVQNMNNAG |
| | | DKWSAFLKEQSTLAQMY |
| 11 | 59 | AKTFLDKFNHEAEDLFYQSSLASWNYNTNITEENVQNMNN |
| | | AGDKWSAFLKEQSTLAQMY |
| 12 | 60 | QAKTFLDKFNHEAEDLFYQSSLASWNYNTNITEENVQNMN |
| | | NAGDKWSAFLKEQSTLAQMY |
| 13 | 62 | QAKTFLDKFNHEAEDLFYQSSLASWNYNTNITEENVQNMN |
| | | NAGDKWSAFLKEQSTLAQMYPL |
| 14 | 70 | STIEEQAKTFLDKENHEAEDLEYQSSLASWNYNTNITEENV |
| | | QNIMNNAGDKWSAFLKEQSTLAQMYPLQEI |
| 15 | 70 | TDAMVDQAWDAQRIFKEAEKFFVSVGLPNMTQGFWENSM |
| | | LTDPGNVQKAVCHPTAWDLGKGDFRILMCTK |
| | | VT |
| 16 | 73 | STIEEQAKTFLDKENHEAEDLEYQSSLASWNYNTNITEENV |
| | | QNMNNAGDKWSAFLKEQSTLAQMYPLQEI |
| | | QNL |
| 17 | 78 | STIEEQAKTFLDKENHEAEDLEYQSSLASWNYNTNITEENV |
| | | QNMNNAGDKWSAFLKEQSTLAQMYPLQEI |
| | | QNLTVKLQ |
| 18 | 79 | STIEEQAKTFLDKENHEAEDLEYQSSLASWNYNTNITEENV |
| | | QNMNNAGDKWSAFLKEQSTLAQMYPLQEI |
| | | QNLTVKLQL |
| 19 | 81 | STIEEQAKTFLDKENHEAEDLEYQSSLASWNYNTNITEENV |
| | | QNMNNAGDKWSAFLKEQSTLAQMYPLQEIQNLTVKLQLQA |
| 20 | 83 | STIEEQAKTFLDKENHEAEDLEYQSSLASWNYNTNITEENV |
| | | QNMNNAGDKWSAFLKEQSTLAQMYPLQEIQNLTVKLQLQALQ |
| 21 | 85 | STIEEQAKTFLDKENHEAEDLEYQSSLASWNYNTNITEENV |
| | | QNMNNAGDKWSAFLKEQSTLAQMYPLQEI |
| | | NLTVKLQLQALQQN |
| 22 | 115 | STIEEQAKTFLDKFNHEAEDLFYQSSLASWNYNTNITEENV |
| | | QNMNNAGDKWSAFLKEQSTLAQMYPLQEIQNLTVKLQLQ |
| | | ALQQNGSSVLSEDKSKRLNTILNTMSTIYSTGKVC |
| 23 | 126 | AMVDQAWDAQRIFKEAEKFFVSVGLPNMTQGFWENSMLT |
| | | DPGNVQKAVCHPTAWDLGKGDFRILMCTKVT |
| | | MDDFLTAHHEMGHIQYDMAYAAQPFLLRNGANEGFHEAV |
| | | GEIMSLSAATPKHLKSIGL |
| 24 | 124 | STIEEQAKTFLDKFNHEAEDLFYQSSLASWNYNTNITEENV |
| | | QNMNNAGDKWSAFLKEQSTLAQMYPLQEIQNLTVKLQLQ |
| | | ALQQNGSSVLSEDKSKRLNTILNTMSTIYSTGKVCNPDNPQECL |
| 25 | 129 | VTDAMVDQAWDAQRIFKEAEKFFVSVGLPNMTQGFWENS |
| | | MLTDPGNVQKAVCHPTAWDLGKGDFRILMCT |
| | | KVTMDDFLTAHHEMGHIQYDMAYAAQPFLLRNGANEGFH |
| | | EAVGEIIVISLSAATPKHLKSIGL |
| 26 | 134 | STIEEQAKTFLDKFNHEAEDLFYQSSLASWNYNTNITEENV |
| | | QNMNNAGDKWSAFLKEQSTLAQMYPLQEIQNLTVKLQLQ |
| | | ALQQNGSSVLSEDKSKRLNTILNTMSTIYSTGKVCNPDNPQ |
| | | ECLLLEPGLNEIM |
| 27 | 148 | STIEEQAKTFLDKFNHEAEDLFYQSSLASWNYNTNITEENV |
| | | QNMNNAGDKWSAFLKEQSTLAQMYPLQEI |
| | | QNLTVKLQLQALQQNGSSVLSEDKSKRLNTILNTMSTIYST |
| | | GKVCNPDNPQECLLLEPGLNEEVIANSLDY |
| | | NERLWAWE |
| 28 | 597 | STIEEQAKTFLDKENHEAEDLEYQSSLASWNYNTNITEENV |
| | | QNMNNAGDKWSAFLKEQSTLAQMYPLQEIQNLTVKLQLQ |
| | | ALQQNGSSVLSEDKSKRLNTILNTMSTIYSTGKVCNPDNPQ |
| | | ECLLLEPGLNEINIANSLDYNERLWAWESWRSEVGKQLRPL |
| | | YEEYVVLKNEMARANHYEDYGDYWRGDYEVNGVDGYD |
| | | YSRGQLIEDVEHTFEEIKPLYEHLHAYVRAKLMNAYPSYISP |
| | | IGCLPAHLLGDMWGRFWTNLYSLTVPFGQKPNIDVTDAMV |
| | | DQAWDAQRIFKEAEKFFVSVGLPNMTQGFWENSMLTDPG |
| | | NVQKAVCAPTAWDLGKGDFRILMCTKVTMDDFLTAHHEM |
| | | GHIQYDMAYAAQPFLLRNGANEGFHEAVGEIMSLSAATPK |
| | | HLKSIGLLSPDFQEDNETEINFLLKQALTIVGTLPFTYMLEK |
| | | WRWMVFKGEIPKDQWMKKWWEMKREIVGVVEPVPHDET |
| | | YCDPASLFHVSNDYSFIRYYTRTLYQFQFQEALCQAAKHEG |
| | | PLHKCDISNSTEAGQKLFNMLRLGKSEPWTLALENVVGAK |
| | | NMNVRPLLNYFEPLFTWLKDQNKNSFVGWSTDWSPYAD |
| 29 | 597 | STIEEQAKTFLDKENHEAEDLEYQSSLASWNYNTNITEENV |
| | | QNMNNAGDKWSAFLKEQSTLAQMYPLQEIQNLTVKLQLQ |
| | | ALQQNGSSVLSEDKSKRLNTILNTMSTIYSTGKVCNPDNPQ |
| | | ECLLLEPGLNEINIANSLDYNERLWAWESWRSEVGKQLRPL |
| | | YEEYVVLKNEMARANHYEDYGDYWRGDYEVNGVDGYD |
| | | YSRGQLIEDVEHTFEEIKPLYEHLHAYVRAKLMNAYPSYISP |
| | | IGCLPAHLLGDMWGRFWTNLYSLTVPFGQKPNIDVTDAMV |
| | | DQAWDAQRIFKEAEKFFVSVGLPNMTQGFWENSMLTDPG |
| | | NVQKAVCLPTAWDLGKGDFRILMCTKVTMDDFLTAHHEM |
| | | GHIQYDMAYAAQPFLLRNGANEGFHEAVGEIMSLSAATPK |
| | | HLKSIGLLSPDFQEDNETEINFLLKQALTIVGTLPFTYMLEK |
| | | WRWMVFKGEIPKDQWMKKWWEMKREIVGVVEPVPHDET |
| | | YCDPASLFHVSNDYSFIRYYTRTLYQFQFQEALCQAAKHEG |
| | | PLHKCDISNSTEAGQKLFNMLRLGKSEPWTLALENVVGAK |
| | | NMNVRPLLNYFEPLFTWLKDQNKNSFVGWSTDWSPYAD |
| 30 | 597 | STIEEQAKTFLDKENHEAEDLEYQSSLASWNYNTNITEENV |
| | | QNMNNAGDKWSAFLKEQSTLAQMYPLQEIQNLTVKLQLQ |
| | | ALQQNGSSVLSEDKSKRLNTILNTMSTIYSTGKVCNPDNPQ |
| | | ECLLLEPGLNEEVIANSLDYNERLWAWESWRSEVGKQLRPL |
| | | YEEYVVLKNEMARANHYEDYGDYWRGDYEVNGVDGYD |
| | | YSRGQLIEDVEHTFEEIKPLYEHLHAYVRAKLMNAYPSYISP |
| | | IGCLPAHLLGDMWGRFWTNLYSLTVPFGQKPNIDVTDAMV |
| | | DQAWDAQRIFKEAEKFFVSVGLPNMTQGFWENSMLTDPG |
| | | NVQKAVCHPTAWDLGKGDFRILMCTKVTMDDFLTAHHEM |
| | | GHIQYDMAYAAQPFLLRNGANEGFHEAVGEIMSLSAATPK |
| | | HLKSIGLLSPDFQEDNETEINFLLKQALTIVGTLPFTYMLEK |
| | | WRWMVFKGEIPKDQWMKKWWEMKREIVGVVEPVPHDET |
| | | YCDPASLFAVSNDYSFIRYYTRTLYQFQFQEALCQAAKHEG |
| | | PLHKCDISNSTEAGQKLFNMLRLGKSEPWTLALENVVGAK |
| | | NMNVRPLLNYFEPLFTWLKDQNKNSFVGWSTDWSPYAD |
| 31 | 597 | STIEEQAKTFLDKENHEAEDLEYQSSLASWNYNTNITEENV |
| | | QNMNNAGDKWSAFLKEQSTLAQMYPLQEIQNLTVKLQLQ |
| | | ALQQNGSSVLSEDKSKRLNTILNTMSTIYSTGKVCNPDNPQ |
| | | ECLLLEPGLNEEVIANSLDYNERLWAWESWRSEVGKQLRPL |
| | | YEEYVVLKNEMARANHYEDYGDYWRGDYEVNGVDGYD |
| | | YSRGQLIEDVEHTFEEIKPLYEHLHAYVRAKLMNAYPSYISP |
| | | IGCLPAHLLGDMWGRFWTNLYSLTVPFGQKPNIDVTDAMV |
| | | DQAWDAQRIFKEAEKFFVSVGLPNMTQGFWENSMLTDPG |
| | | NVQKAVCHPTAWDLGKGDFRILMCTKVTMDDFLTAHHEM |
| | | GHIQYDMAYAAQPFLLRNGANEGFHEAVGEIMSLSAATPK |
| | | HLKSIGLLSPDFQEDNETEINFLLKQALTIVGTLPFTYMLEK |
| | | WRWMVFKGEIPKDQWMKKWWEMKREIVGVVEPVPHDET |
| | | YCDPASLFLVSNDYSFIRYYTRTLYQFQFQEALCQAAKHEG |
| | | PLHKCDISNSTEAGQKLFNMLRLGKSEPWTLALENVVGAK |
| | | NMNVRPLLNYFEPLFTWLKDQNKNSFVGWSTDWSPYAD |

### EXAMPLES

### Example 1

### Computationally Optimized Peptides Targeting the SARS-CoV- 2 RBD

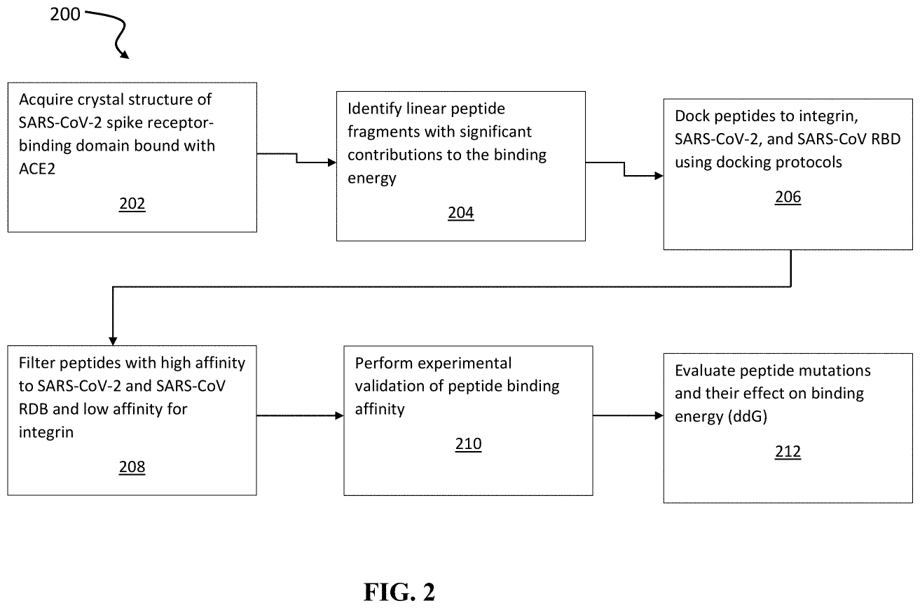

Referring now to FIG. 2, Example 1 demonstrates a method 200 for generating and evaluating engineered targeting peptides, according to the present disclosure. This example further assesses whether an RBD-protein targeting peptide, according to the present disclosure, exhibits cross-binding affinity toward previous spike proteins for which a known structure exists, thus allowing determination of the peptides tolerance to viral evolution.

A structure of the SARS-CoV-2 RBD bound to sACE2 was retrieved from the Protein Data Bank (PDB 6MOJ) 202. The PeptiDerive protocol in the Rosetta protein modeling software was used to generate truncated linear sACE2 peptide segments between 10 and 150 amino acids with significant binding energy compared to that of the full SARS-CoV-2 RBD- sACE2 interaction 204. To analyze the conformational entropy of the peptide segments in the binding pocket, both the FlexPepDock and Protein-Protein protocols were employed to dock the peptides to the original RBD 206. To ensure tolerance to potential mutations in the RBD, peptides were docked with optimal binding energies against the divergent 2003 SARS-CoV RBD bound with ACE2 (PDB 2AJF). Peptides that demonstrated highest binding energy for SARS-CoV and SARS- CoV-2 RBD were then docked against the a5131 integrin ectodomain (PDB 3VI4) to identify weak off-target binders 208. Finally, as bounded ligands may alter the native conformation of proteins, the conformational stability of the candidate peptides as monomers was confirmed to rule out any destabilizing factors in their unbound state 210. After applying these filters, 26 candidate peptides (corresponding to SEQ. ID. 1-2 and 4-27) were selected from a total list of 188 initial peptides 212.

### Example 2

### Targeted Degradation of RBD

TRIM21 is an E3 ubiquitin ligase that binds with high affinity to the Fc domain of antibodies and recruits the ubiquitin-proteasome system to degrade targeted proteins. Recently, the Trim-Away technique was developed for acute and rapid degradation of endogenous proteins, by co-expressing TRIM21 with an anti-target anti- body [Clift, D. et al. A method for the acute and rapid degradation of endogenous proteins. Cell 171, 1692-1706.e18 (2017).]. By fusing the Fc domain to the C-terminus of candidate peptides and co-expressing TRIM21, degradation of the RBD fused to a stable fluorescent marker, such as superfolder GFP9 (RBD-sfGFP), in human HEK293T cells can be mediated using a simple plasmid-based assay. The two most compact candidate peptides from Example 1 were used, an 18-mer (corresponding to SEQ. ID. 1) and 23-mer (corresponding to SEQ. ID. 2) derived from the ACE2 peptidase domain al helix, which is composed entirely of proteinogenic amino acids, as well as the candidate peptide computationally predicted to have highest binding affinity to the RBD (a 148-mer, corresponding to SEQ. ID. 27), for testing alongside sACE2. A recently-engineered 23-mer peptide (corresponding to SEQ. ID. 3) from Zhang, et al., purporting to have strong RBD-binding capabilities [Zhang, G., Pomplun, S., Loftis, A. R., Loas, A. & Pentelute, B. L. The first-inclass peptide binder to the SARS-CoV-2 spike protein. bioRxiv https://doi.org/10.1101/2020.03.19.999318 (2020).], was also tested. Five days post transfection, the degradation of the RBD-sfGFP complex was analyzed by flow cytometry. After confirming negligible baseline depletion of GFP+ signal with and without exogenous TRIM21 expression, as well as no off-target degradation of sfGFP unbound to the RBD, over 30% reduction of GFP+ cells treated with full- length sACE2 fused to Fc and co-expressed with TRIM21 was observed, as compared to the RBD-sfGFP-only control. Of the tested peptides, only the 23-mer demonstrated comparable levels of degradation, with ˜20% reduction in GFP+ cells.

### Example 3

### Engineering of an Optimal Peptide-Based Degradation Architecture

Recently, deep mutational scans have been conducted on sACE2 to identify variants with higher binding affinity to the RBD of SARS-CoV-229. Similarly, a complete single point mutational scan was conducted for all 23 positions in the peptide using the ddG-backrub script in Rosetta to identify mutants with improved binding affinity [Barlow, K. A. et al. Flex ddG: Rosetta ensemble-based estimation of changes in protein-protein binding affinity upon mutation. J. Phys. Chem. B 122, 5389-5399 (2018).]. For each mutation, 30,000 backrub trials were performed to sample conformational diversity. The top eight mutations predicted by this protocol were used for an experimental assay, along with the top eight mutations predicted using an Rosetta energy function optimized for predicting the effect of mutations on protein-protein binding, as well as the top eight mutational sites within the 23-mer sequence (corresponding to SEQ. ID. 2) from deep mutational scans of sACE2. Results in the subsequent TRIM21 assay identified A2N, derived from the original Rosetta energy function, as the optimal mutation in the 23-mer peptide, which achieved over 50% depletion of GFP+ cells, improving on both the sACE2 and 23-mer architecture as well as that of a previously optimized full- length mutant, sACE2v2.4.

Previous works have attempted to redirect E3 ubiquitin ligases by replacing their natural protein binding domains with those targeting specific proteins. In 2014, Portnoff, et al. reprogrammed the substrate specificity of a modular human E3 ubiquitin ligase called CHIP (carboxyl-terminus of Hsc70-interacting protein) by replacing its natural substrate-binding domain with designer binding proteins to generate optimized “ubiquibodies” or uAbs. To engineer a single construct that can mediate SARS-CoV-2 degradation without the need for trans expression of TRIM21, the RBD-binding proteins were fused to the CHIPΔTPR modified E3 ubiquitin ligase domain. After co-transfection in HEK293T cells with the RBD-sfGFP complex, it was observed that the 23-mer (A2N) mutant peptide maintained equivalent levels of degradation between the TRIM21 and CHIPATPR fusion architecture, and was more potent than that of sACE2, sACE2v2.4, and the original 23-mer. Full-length sACE2 and optimized mutant sACE2v2.4, in particular, were less compatible with the CHIPATPR fusion architecture, possibly owing to steric occlusion caused by the larger size of the initial complex.

### Example 4

### Inhibition of Infection-Competent Viral Production

The efficacy of the 23-mer (A2N)-CHIPATPR fusion against viruses pseudotyped with the SARS-CoV-2 S protein was assessed. A plasmid encoding the construct was introduced during lentiviral production with a ZsGreen expression plasmid, lentivirus packaging plasmid, and an envelope protein plasmid encoding the full-length S protein, rather than just the RBD. After viral supernatant recovery, HEK293T cells expressing doxycycline-induced hACE2 were infected, and quantified infection as the percentage of ZsGreen+ cells by flow cytometry. Results showed that the 23-mer (A2N)-CHIPΔTPR fusion reduces the infection rate of the pseudovirus by ˜60%, in agreement with our RBD-sfGFP degradation data.

Other possible mechanisms of action for the peptide variant were tested, namely competitive interception of S protein-pseudotyped virus prior to cellular entry. The 23-mer (A2N) peptide was synthesized, and the pseudoviral assay was repeared in its presence or absence at a standard dosage (1 μg/ml) [Monteil, V. et al. Inhibition of SARS-CoV-2 infections in engineered human tissues using clinical-grade soluble human ACE2. Cell 181, 905-913.e7 (2020).]. Minimal difference in pseudoviral infection competency was observed with the addition of the exogenous peptide in all tested experimental conditions, thus suggesting that intracellular delivery of the 23-mer (A2N)-CHIPΔTPR fusion may be the optimal modality for the peptide to inhibit viral infection.

At least the following aspects, implementations, modifications, and applications of the described technology are contemplated by the inventors and areconsidered to be aspects of the present disclosure: (1) the engineered sACE2-derived protein sequences listed in Table 1; and (2) the uses of the sACE2-derived protein sequences listed in Table 1 as biologics for COVID-19.

While certain embodiments of the present disclosure are discussed herein, many other implementations will occur to one of ordinary skill in the art and are all within the scope of the invention. Each of the various embodiments described above may be combined with other described embodiments in order to provide multiple features.

Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the artare therefore also considered to be within the scope of the present invention.

1. An engineered peptide capable of binding to a biological molecule for viral inhibition, the engineered peptide comprising:
an engineered sACE2 peptide variant having an amino acid sequence comprising QAKTFLDKFNHEAEDLFY or AMVDQAWDAQRIFKEAEKFFVSVGLPNMTQGFWENSMLTDPGNVQKAVCHPTAWDL GKGDFRILMCTKVT.

2. The engineered peptide of claim 1, wherein the amino acid sequence of the engineered sACE2 peptide variant is selected from the group consisting of sACE2 peptide variant 1 (SEQ ID NO:1), sACE2 peptide variant 2 (SEQ ID NO:2), sACE2 peptide variant 3 (SEQ ID NO:3), sACE2 peptide variant 4 (SEQ ID NO:4), sACE2 peptide variant 5 (SEQ ID NO:5), sACE2 peptide variant 6 (SEQ ID NO:6), sACE2 peptide variant 7 (SEQ ID NO:7), sACE2 peptide variant 8 (SEQ ID NO:8), sACE2 peptide variant 9 (SEQ ID NO:9), sACE2 peptide variant 10 (SEQ ID NO:10), sACE2 peptide variant 11 (SEQ ID NO:11), sACE2 peptide variant 12 (SEQ ID NO:12), sACE2 peptide variant 13 (SEQ ID NO:13), sACE2 peptide variant 14 (SEQ ID NO:14), sACE2 peptide variant 16 (SEQ ID NO:16), sACE2 peptide variant 17 (SEQ ID NO:17), sACE2 peptide variant 18 (SEQ ID NO:18), sACE2 peptide variant 19 (SEQ ID NO:19), sACE2 peptide variant 20 (SEQ ID NO:20), sACE2 peptide variant 21 (SEQ ID NO:21), sACE2 peptide variant 22 (SEQ ID NO:22), sACE2 peptide variant 24 (SEQ ID NO:24) sACE2 peptide variant 26 (SEQ ID NO:26), sACE2 peptide variant 27 (SEQ ID NO:27), sACE2 peptide variant 28 (SEQ ID NO:28), sACE2 peptide variant 29 (SEQ ID NO:29), sACE2 peptide variant 30 (SEQ ID NO:30), and sACE2 peptide variant 31 (SEQ ID NO:31).

3. The engineered peptide of claim 2, wherein the engineered sACE2 peptide variant sACE2 peptide variant 2 (SEQ ID NO:2).

4. The engineered peptide of claim 1, wherein the engineered sACE2 peptide variant is between 18 and 30 amino acids in length.

5. The engineered peptide of claim 1, wherein the amino acid sequence of the engineered sACE2 peptide variant is selected from the group consisting of sACE2 peptide variant 15 (SEQ ID NO:15), sACE2 peptide variant 23 (SEQ ID NO:23) and sACE2 peptide variant 25 (SEQ ID NO:25).

6. The engineered peptide of claim 1, wherein the engineered sACE2 peptide variant binds a spike protein receptor.

7. The engineered peptide of claim 6, wherein the spike protein receptor is part of a viral envelope.

8. The engineered peptide of claim 6, wherein the spike protein receptor is part of a coronavirus.

9. The engineered peptide of claim 8, wherein the coronavirus is SARS-CoV-2.

10. The engineered peptide of claim 1, wherein the engineered sACE2 peptide variant is fused to a ubiquitin ligase recruiting domain.

11. The engineered peptide of claim 10, wherein the engineered sACE2 peptide variant has a C-terminus, and the ubiquitin ligase recruiting domain is fused to the C-terminus.

12. A pharmaceutical composition comprising the engineered peptide of claim 1.

13. The pharmaceutical composition of claim 12, further comprising two or more of the engineered peptides of claim 1, for simultaneous, sequential or separate administration.

14. The pharmaceutical composition of claim 12, wherein the engineered peptide is capable of binding a spike protein receptor.

15. The pharmaceutical composition of claim 14, wherein the spike protein receptor is part of a coronavirus.

16. The pharmaceutical composition of claim 15, wherein the coronavirus is SARS-CoV-2.

17. A method of treating a viral infection in a subject using a computationally engineered peptide for viral targeting, comprising:
providing a pharmaceutical composition to the subject, the pharmaceutical composition comprising the computationally engineered peptide, wherein the computationally engineered peptide comprises an engineered sACE2 peptide variant having an amino acid sequence comprising QAKTFLDKENHEAE5301DLEY or AMVDQAWDAQRIFKEAEKFFVSVGLPNMTQGFWENSMLTDPGNVQKAVCHPTAWDL GKGDFRILMCTKVT.

18. The method of claim 17, wherein the engineered sACE2 peptide variant sACE2 peptide variant 2 (SEQ ID NO:2).

19. The method of claim 17, wherein the receptor binding domain target is a spike protein receptor.

20. The method of claim 17, wherein the spike protein receptor is part of a coronavirus and the coronavirus is SARS-CoV-2.


SEQUENCE LISTING (31 sequences)

SEQ ID NO: 1 - protein, 18 aa, Artificial Sequence
  computationally truncated and engineered human ACE2 receptor sequence
QAKTFLDKFNHEAEDLFY

SEQ ID NO: 2 - protein, 23 aa, Artificial Sequence
  computationally truncated and engineered human ACE2 receptor sequence
QAKTFLDKFNHEAEDLFYQSSLA

SEQ ID NO: 3 - protein, 23 aa, Artificial Sequence
  computationally truncated and engineered human ACE2 receptor sequence
IEEQAKTFLDKFNHEAEDLFYQS

SEQ ID NO: 4 - protein, 25 aa, Artificial Sequence
  computationally truncated and engineered human ACE2 receptor sequence
STIEEQAKTFLDKFNHEAEDLFYQS

SEQ ID NO: 5 - protein, 28 aa, Artificial Sequence
  computationally truncated and engineered human ACE2 receptor sequence
STIEEQAKTFLDKFNHEAEDLFYQSSLA

SEQ ID NO: 6 - protein, 29 aa, Artificial Sequence
  computationally truncated and engineered human ACE2 receptor sequence
STIEEQAKTFLDKFNHEAEDLFYQSSLAS

SEQ ID NO: 7 - protein, 30 aa, Artificial Sequence
  computationally truncated and engineered human ACE2 receptor sequence
STIEEQAKTFLDKFNHEAEDLFYQSSLASW

SEQ ID NO: 8 - protein, 44 aa, Artificial Sequence
  computationally truncated and engineered human ACE2 receptor sequence
STIEEQAKTFLDKFNHEAEDLFYQSSLASWNYNTNITEENVQNM

SEQ ID NO: 9 - protein, 56 aa, Artificial Sequence
  computationally truncated and engineered human ACE2 receptor sequence
QAKTFLDKFNHEAEDLFYQSSLASWNYNTNITEENVQNMNNAGDKWSAFLKEQSTL

SEQ ID NO: 10 - protein, 57 aa, Artificial Sequence
  computationally truncated and engineered human ACE2 receptor sequence
TFLDKFNHEAEDLFYQSSLASWNYNTNITEENVQNMNNAGDKWSAFLKEQSTLAQMY

SEQ ID NO: 11 - protein, 59 aa, Artificial Sequence
  computationally truncated and engineered human ACE2 receptor sequence
AKTFLDKFNHEAEDLFYQSSLASWNYNTNITEENVQNMNNAGDKWSAFLKEQSTLAQMY

SEQ ID NO: 12 - protein, 60 aa, Artificial Sequence
  computationally truncated and engineered human ACE2 receptor sequence
QAKTFLDKFNHEAEDLFYQSSLASWNYNTNITEENVQNMNNAGDKWSAFLKEQSTLAQMY

SEQ ID NO: 13 - protein, 62 aa, Artificial Sequence
  computationally truncated and engineered human ACE2 receptor sequence
QAKTFLDKFNHEAEDLFYQSSLASWNYNTNITEENVQNMNNAGDKWSAFLKEQSTLAQMYPL

SEQ ID NO: 14 - protein, 70 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEI

SEQ ID NO: 15 - protein, 72 aa, Artificial Sequence
  computationally truncated and engineered human ACE2 receptor sequence
TDAMVDQAWDAQRIFKEAEKFFVSVGLPNMTQGFWENSMLTDPGNVQKAVCHPTAWDLGKGDFRILMCTKVT

SEQ ID NO: 16 - protein, 73 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEIQNL

SEQ ID NO: 17 - protein, 78 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEIQNLTVKLQ

SEQ ID NO: 18 - protein, 79 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEIQNLTVKLQL

SEQ ID NO: 19 - protein, 81 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEIQNLTVKLQLQA

SEQ ID NO: 20 - protein, 83 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEIQNLTVKLQLQALQ

SEQ ID NO: 21 - protein, 85 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEIQNLTVKLQLQALQQN

SEQ ID NO: 22 - protein, 115 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVC

SEQ ID NO: 23 - protein, 128 aa, Artificial Sequence
  computationally truncated and engineered human ACE2 receptor sequence
AMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGL

SEQ ID NO: 24 - protein, 124 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECL

SEQ ID NO: 25 - protein, 131 aa, Artificial Sequence
  computationally truncated and engineered human ACE2 receptor sequence
VTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGL

SEQ ID NO: 26 - protein, 134 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIM

SEQ ID NO: 27 - protein, 148 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWE

SEQ ID NO: 28 - protein, 597 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCAPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 29 - protein, 597 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 30 - protein, 597 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFAVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 31 - protein, 597 aa, Artificial Sequence
  computationally truncated and engineered human ACE2 receptor sequence
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFLVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD